(19) United States
(12) Patent Application Publication
(10) Pub. No.: US 2017/0347772 A1
(43) Pub. Date: Dec. 7, 2017

(54) COMPACT CONTAINER HAVING DISCHARGE PLATE MADE OF CERAMIC

(71) Applicant: Sungil KANG, Seongnam-si (KR)

(72) Inventor: Sungil KANG, Seongnam-si (KR)

(21) Appl. No.: 15/537,003

(22) PCT Filed: Dec. 16, 2015
(86) PCT No.: PCT/KR2015/013824

§ 371 Date: Jun. 16, 2017

(30) Foreign Application Priority Data
Dec. 19, 2014 (KR) 10-2014-0184313

(51) Int. Cl. A45D 40/00; A45D 40/22; A45D 42/02; A61L 2/23; A61L 2/238; C04B 35/08; B01J 20/12; B01J 20/20; C04B 35/58; C04B 35/56; C04B 35/48; A45D 34/00; C04B 35/10; A61L 9/014

(52) U.S. Cl. CPC A45D 40/0075; C04B 35/10; A45D 40/222; A45D 42/02; A61L 2/23; A61L 2/238; A61L 9/014; B01J 20/12; B01J 20/20; C04B 35/58085; C04B 35/58; C04B 35/56; C04B 35/48; A45D 34/00; C04B 35/08; A45D 2040/223; A61L 2209/22; C04B 2235/3205; C04B 2235/3217; C04B 2235/3244; C04B 2235/3817; C04B 2235/3852; C04B 2235/3891

(57) ABSTRACT

The present invention relates to a compact container having a discharge plate made of ceramic, and more specifically, a compact container having a discharge plate made of ceramic, wherein, since the discharge plate is made of ceramic, the discharge plate is not to be worn out when a user spreads gel contents on the discharge plate and impregnates the puff with the gel contents. The ceramic which forms the discharge plate has high hardness and excellent heat resistance and wear resistance, thereby enabling long-time use. Cosmetic is allowed to exert antibacterial and antiseptic actions and heavy metal-removing and deodorizing functions when passing through the discharge plate. In addition, since the discharge plate is made of ceramic, the discharge plate is not bent and is lowered down with the entire part thereof balanced, even when the user presses the discharge plate for use of the cosmetic.

### CROSS-REFERENCE TO RELATED APPLICATION

This application claims the benefit of Korean application No. 10-2014-0184313, filed on Dec. 19, 2014 with the Korean Intellectual Property Office, the disclosure of which is incorporated herein by reference.

### TECHNICAL FIELD

The present invention relates to a compact container having a discharge plate made of ceramic, and more specifically, a compact container having a discharge plate made of ceramic, wherein, since the discharge plate is made of ceramic, the discharge plate is not to be worn out when a user spreads gel contents on the discharge plate and impregnates the puff with the gel contents. The ceramic which forms the discharge plate has high hardness and excellent heat resistance and wear resistance, thereby enabling long-time use. Cosmetic is allowed to exert antibacterial and antiseptic actions and heavy metal-removing and deodorizing functions when passing through the discharge plate. In addition, since the discharge plate is made of ceramic, the discharge plate is not bent and is lowered down with the entire part thereof balanced, even when the user presses the discharge plate for use of the cosmetic.

### BACKGROUND ART

Color cosmetics are used for the purpose of beautifully expressing a skin by beautifully changing the appearance. The color cosmetics are divided into a base makeup used for uniformizing the skin color and covering the defects, and a point makeup for partially enhancing the three-dimensional feeling such as lips, eyes, and nails. The base makeup is classified into a makeup base, a foundation, a powder, and so on, and the point makeup is classified into a lipstick, an eyeliner, a mascara, and so on.

The foundation is divided into a solid foundation, a liquid foundation, and a gel foundation according to the form of a cosmetic material. The solid foundation has a high covering power, however, agglomerates upon correction makeup, and the liquid foundation provides good adhering felling, however, has weak sustainability. Accordingly, many consumers recently prefer the gel foundation having considerable durability and good adhering feeling when applying the gel foundation onto the skin.

Thus, the development of a container for the gel foundation is also required. In general, the gel foundation is filled into a glass container or a tube container, and used in the manner that the gel foundation is drawn or squeezed on a hand of the user and applied onto the skin using a puff or a hand.

However, the related art causes the inconvenience in that the user is required to wash the cosmetic material put on the hands whenever using the cosmetic material, the cosmetic material is wasted since the cosmetic material put on the hands is washed as described above.

To solve the problems as the above, as shown in FIG. 1, Korean Patent Registration No. 10-1471339 has been disclosed, in which the gel cosmetic material filled in a compact container, so that the cosmetic material can be used without putting the cosmetic material on a hand, and it is convenient to carry the cosmetic material.

However, in general, the gel cosmetic material is discharged through a discharge plate, the discharged cosmetic material is rubbed and spread by a puff, and uniformly impregnated into the puff, thus the gel cosmetic material is applied to a skin to makeup. According to the related art, because the discharge plate is formed of metal, fine stone powder, which is a powder raw material of the gel cosmetic material, wears out a surface of the metal discharge plate when the gel cosmetic material is rubbed and spread on the metal discharge plate by using the puff, thus malodor generates upon wearing of the metal, and the worn metal powder discolors the cosmetic material, and is applied to the skin with the cosmetic material, thereby causing skin troubles.

### DISCLOSURE

### Technical Problem

To solve the problems as the above, the present invention provides a compact container having a discharge plate made of a ceramic material, in which the discharge plate is formed of a ceramic material so as to prevent the discharge plate from being worn out when a user spreads a gel cosmetic material on the discharge plate and impregnates the puff with the gel cosmetic material, the ceramic material forming the discharge plate has high hardness, and excellent heat resistance and wear resistance so as to use the discharge plate for a long time, and antibacterial and antiseptic actions and heavy metal-removing and deodorizing functions are exerted to the cosmetic material when the cosmetic material passes through the discharge plate.

In addition, the object of the present invention is to provide a compact container having a discharge plate made of a ceramic material, in which the discharge plate is formed of a ceramic material, so that the discharge plate entirely horizontally moves down while being prevented from bending when the user pressurizes the discharge plate to use the cosmetic material.

### Technical Solution

There is provided a compact container of the present invention including an outer container (10) formed on one side thereof with a button (11), and an outer container lid (20) hinged to one side of the outer container (10) to be opened and closed, includes: an inner container (30) mounted inside the outer container (10); an inner container cover (40) coupled to an upper portion of the inner container (30), and formed at a center thereof with a cosmetic material suction hole (41); a pump (50) mounted on an upper portion of the inner container cover (40) to pump the cosmetic material; and a discharge plate (60) formed of a ceramic material, coupled to an upper side of the inner container cover (40) so as to be vertically movable, coupled to a top of the pump (50), formed therein with a plurality of cosmetic material exhausting holes (61), having an upper portion exposed to the outside to make contact with a puff (P) by a user, having high hardness, heat resistance and wear resistance, and having antibacterial, antiseptic, heavy metal-removing and deodorizing functions.

In addition, the discharge plate (60) has a thickness of 0.5 mm to 3.0 mm.

In addition, the cosmetic material exhausting hole (61) of the discharge plate (60) has a diameter of 0.5 mm to 1.5 mm.

In addition, the cosmetic material exhausting hole (61) of the discharge plate (60) has a diameter which gradually increases from a center toward an outer side of the discharge plate (60).

In addition, at least one of a first sealing ring (31) and a second sealing ring (32) is further coupled between an upper end of the inner container (30) and the inner container cover (40).

In addition, a cosmetic material storage membrane (33) in the form of a soft thin membrane is formed on an inner side of the inner container (30).

In addition, the pump (50) is formed at a top end thereof with a cosmetic material diffusion space (58) having a radial shape.

In addition, a mesh (70) is interposed between the pump (50) and the discharge plate (60).

In addition, the mesh (70) is attached to a top end of the pump (50) by a double-sided tape (71).

In addition, an adhesion space (59) attached thereto with the double-sided tape (71) is preferably formed at an outer side of the cosmetic material diffusion space (58) of the pump (50).

### Advantageous Effects

According to the compact container having a discharge plate made of a ceramic material the present invention, the discharge plate is formed of a ceramic material so as to prevent the discharge plate from being worn out when a user spreads a gel cosmetic material on the discharge plate and impregnates the puff with the gel cosmetic material, the ceramic material forming the discharge plate has high hardness, and excellent heat resistance and wear resistance so as to use the discharge plate for a long time, and antibacterial and antiseptic actions and heavy metal-removing and deodorizing functions can be exerted to the cosmetic material when the cosmetic material passes through the discharge plate.

In addition, the discharge plate is formed of a ceramic material, so that the discharge plate can entirely horizontally move down while being prevented from bending when the user presses the discharge plate to use the cosmetic material.

### DESCRIPTION OF DRAWINGS

FIG. 1 shows a compact container filled with a gel cosmetic material.

FIG. 2 is a perspective view showing a compact container having a discharge plate made of a ceramic material according to the present invention.

FIG. 3 is an exploded perspective view showing a compact container having a discharge plate made of a ceramic material according to the present invention.

FIG. 4 is a sectional view showing a compact container having a discharge plate made of a ceramic material according to the present invention.

FIG. 5 is a sectional view showing a state of pressurizing a discharge plate of a compact container having the discharge plate made of a ceramic material according to the present invention.

FIG. 6 is a sectional view showing a state of releasing the pressurization of a compact container having a discharge plate made of a ceramic material according to the present invention.

### BEST MODE

### Mode for Invention

An embodiment of a compact container having a discharge plate made of a ceramic material according to the present invention will be described with reference to the accompanying drawings.

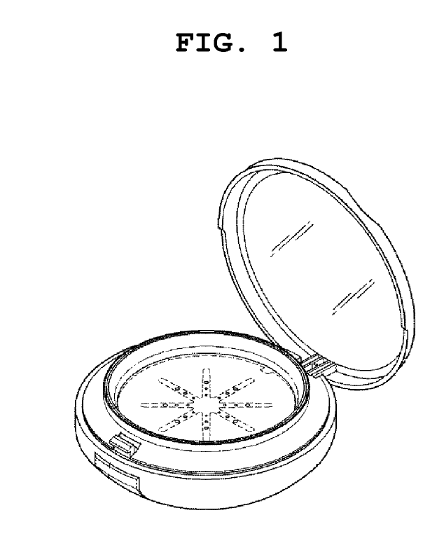

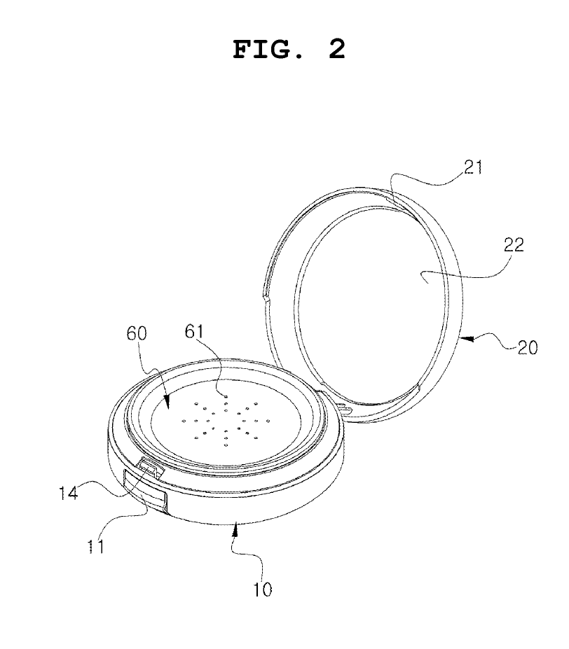

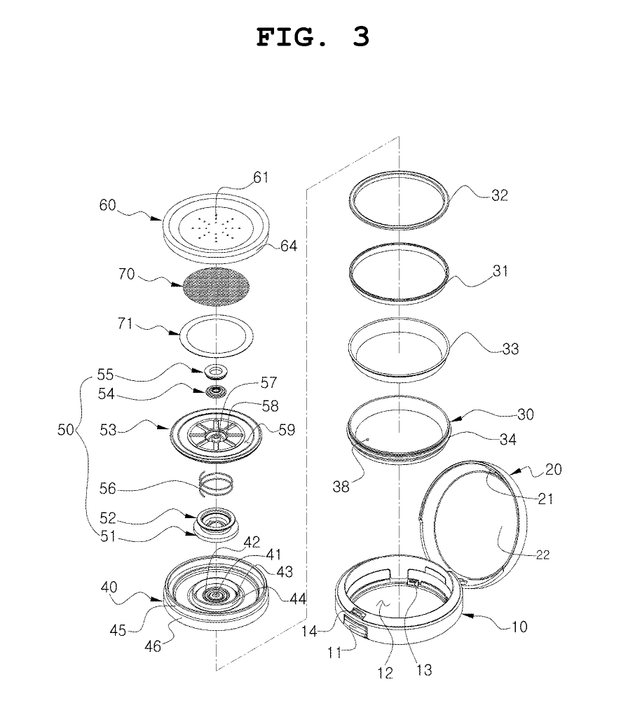

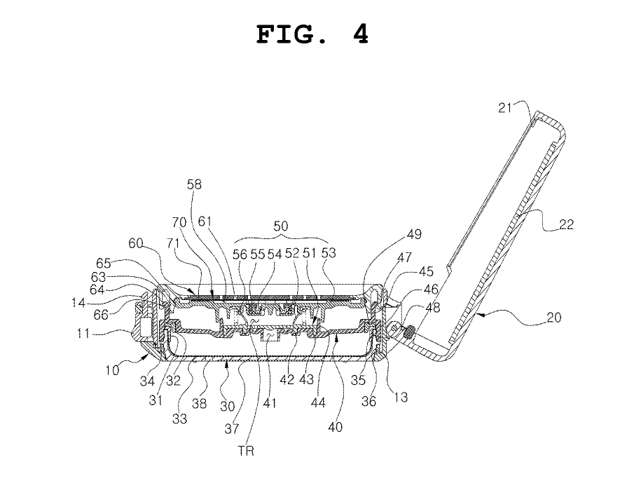

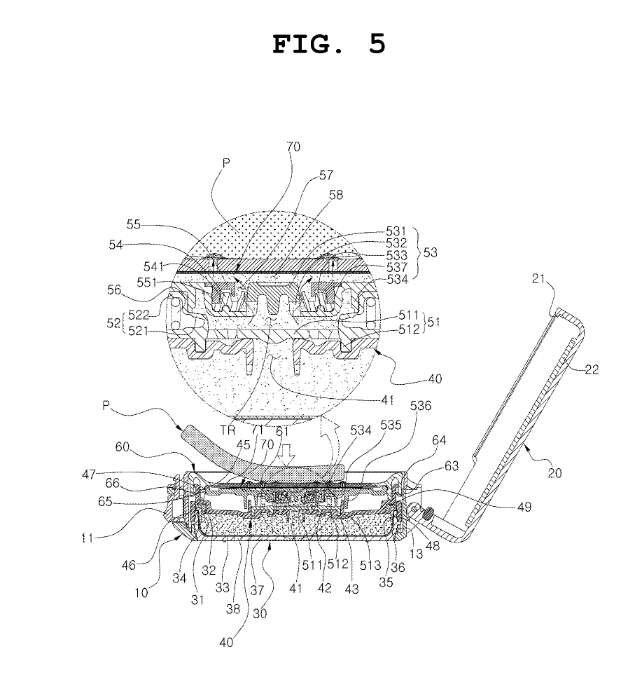

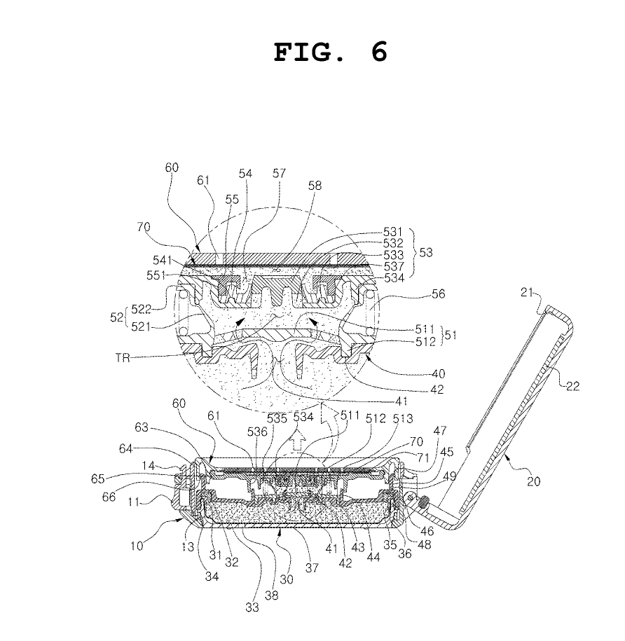

FIG. 1 shows a compact container filled with a gel cosmetic material. FIG. 2 is a perspective view showing a compact container having a discharge plate made of a ceramic material according to the present invention. FIG. 3 is an exploded perspective view showing a compact container having a discharge plate made of a ceramic material according to the present invention. FIG. 4 is a sectional view showing a compact container having a discharge plate made of a ceramic material according to the present invention. FIG. 5 is a sectional view showing a state of pressurizing a discharge plate of a compact container having the discharge plate made of a ceramic material according to the present invention. FIG. 6 is a sectional view showing a state of releasing the pressurization of a compact container having a discharge plate made of a ceramic material according to the present invention.

As shown in FIGS. 3 and 4, the compact container having a discharge plate made of a ceramic material according to an embodiment of the present invention including an outer container 10 formed on one side thereof with a button 11, and an outer container lid 20 hinged to one side of the outer container 10 to be opened and closed, includes: an inner container 30 mounted inside the outer container 10; an inner container cover 40 coupled to an upper portion of the inner container 30, and formed at a center thereof with a cosmetic material suction hole 41; a pump 50 mounted on an upper portion of the inner container cover 40 to pump the cosmetic material; and a discharge plate 60 formed of a ceramic material, coupled to an upper side of the inner container cover 40 so as to be vertically movable, coupled to a top of the pump 50, formed therein with a plurality of cosmetic material exhausting holes 61, having an upper portion exposed to the outside to make contact with a puff P by a user, having high hardness, heat resistance and wear resistance, and having antibacterial, antiseptic, heavy metal-removing and deodorizing functions.

An inner container accommodation space 12 for accommodating the inner container 30 is formed inside the outer container 10, and a mount protrusion 13 is formed on an inner side surface of the outer container 10.

A button 11 is formed on one side of the outer container 10, in which a latching protrusion 14, which is easily retracted upon a pressing action of the user, is integrally formed on an upper side of the button 11.

The outer container lid 20 serves to open and close the outer container 10, and a protrusion-shape hook 21 corresponding to the latching protrusion 14 of the outer container 10 is formed on one side of the outer container lid 20, thereby being fastened to the latching protrusion 14 of the outer container 10.

In addition, the outer container lid 20 is provided therein with a mirror 22, so that the user can easily make up.

An inner container step 34 mounted thereon with the inner container cover 40 is formed on an outer periphery of the inner container 30, and the inner container step 34 is formed on an upper side thereof with a coupling groove 35 coupled to the inner container cover 40, and formed on a lower side thereof with a mount groove 36 coupled to the mount protrusion 13 of the outer container 10. In addition, a bottom surface 37 of the inner container 30 is formed therein with an air flow hole 38 into which external air introduced.

A cosmetic material storage membrane 33 in the form of a soft thin membrane is formed on an inner side of the inner container 30.

Preferably, the cosmetic material storage membrane 33 is formed of at least one of polyethylene phthalate PET resin, polyethylene, polypropylene and thermoplastic elastomer.

At least one of a first sealing ring 31 and a second sealing ring 32 may be further coupled between an upper end of the inner container 30 and the inner container cover 40 to improve sealing performance of the gel cosmetic material accommodated in the inner container 30.

A first pump mount groove 42 and a second pump mount groove 43 mounted thereon with the pump 50 are sequentially spaced apart from each other at a predetermined interval on an outer upper surface of the cosmetic material suction hole 41 of the inner container cover 40.

A first upper extending protrusion wheel 44 upwardly extends from an outer side of the second pump mount groove 43, a second upper extending protrusion wheel 45 is spaced apart from an outer side of first upper extending protrusion wheel 44 at a predetermined interval and upwardly extends, and a lower extending protrusion wheel 46 downwardly extends from an outer side of the second upper extending protrusion wheel 45.

A fastening protrusion 47 is formed on an upper side of the second upper extension protrusion wheel 45, and a coupling protrusion 48 is formed on a lower side of the lower extending protrusion wheel 46, thereby being coupled to the coupling groove 35 of the inner container 30.

In addition, the lower extending protrusion wheel 46 is formed on an inner side thereof with a sealing ring insertion groove 49 into which the first sealing ring 32 and the second sealing ring 33 are inserted.

The pump 50 includes a pump bottom plate 51 formed thereon with a suction valve 511, a pump body 52 integrally extending from an upper side of the pump bottom plate 51, a pump top plate 53 coupled to an upper portion of the pump body 52, a discharging valve 54 coupled to an upper center of the pump top plate 53, a fixation member 55 for fixing the discharging valve 54, and an elastic member 56 mounted on an outer side of the pump body 52.

An elastic suction valve 511 for selectively opening and closing the cosmetic material suction hole 41 of the inner container cover 40 is formed at a center of the pump bottom plate 51.

A first pump mount protrusion 512 and a second pump mount protrusion 513 inserted into the first and second pump mount grooves 42 and 43 of the inner container cover 40 are sequentially spaced apart from each other at a predetermined interval on an outer side of the suction valve 511.

The pump body 52 is integrally formed on a top of the pump bottom plate 51, and includes an elastic support 521 having an elastic cylindrical shape, and a horizontal extension piece 522 horizontally extending from an upper end of the elastic support 521.

A blocking member 531 is formed at a center of the pump top plate 53, a plurality of cosmetic material discharging holes 532 are formed on a lower outer periphery of the blocking member 531, and a top plate mount protrusion 533 mounted thereon with the discharging valve 54 is formed on an outer side of the cosmetic material discharging holes 532.

In addition, a first extension piece 534 is formed on a lower side of the pump top plate 53, a second extension piece 535 and a third extension piece 536 are sequentially formed on an outer side of the first extension piece 534, a horizontal extension piece 522 of the pump body 52 is fitted between the first extension piece 534 and the second extension piece 535, and the third extension piece 536 is located at an outer side the first upper extension protrusion wheel 44 of the container cover 40.

In addition, a pump discharging space 57 filled with the cosmetic material is formed between the outside of the blocking member 531 of the pump top plate 53 and the mesh 70, and the pump discharging space 57 is formed on an inner side thereof with a top plate coupling groove 537 to which the fixation member 55 is coupled.

The discharging valve 54 is formed of an elastic material to selectively open and close the cosmetic material discharging hole 532, while making close contact with or being spaced apart from an outer periphery of the blocking member 531 of the pump top plate 53.

The discharging valve 54 is formed on a lower surface thereof with a discharging valve mount groove 541 coupled to the top plate mount protrusion 533 of the pump top plate 53.

The fixation member 55 is formed on an outer periphery thereof with a fixation member coupling protrusion 551 coupled to the upper plate coupling groove 537 of the pump top plate 53, thereby serving to fix the discharging valve 54.

The elastic member 56 is mounted between an upper surface of the pump bottom plate 51 and the horizontal extension piece 522 of the pump body 52, thereby elastically supporting the horizontal extension piece 522 of the pump body 52 and the pump top plate 53.

Preferably, the suction valve 511 of the pump bottom plate 51, the elastic support 521 of the pump body 52, and the discharging valve 54 are formed of at least one of elastomer, silicone rubber, acrylonitrile-butadiene rubber (NBR), polyethylene (PE), and polypropylene (PP).

In addition, a cosmetic material diffusion space 58 having a radial shape is formed on a top end of the pump top plate 53 of the pump 50, such that the gel cosmetic material widely spreads to be discharged through the discharge plate.

Preferably, an adhesion space 58 attached thereto with a double-sided tape 71 is formed on an outer side of the cosmetic material diffusion space 58 of the pump 50.

An inner wall 63 coupled to the pump top plate 53 of the pump 50 downwardly extends from the discharge plate 60, and an outer wall 64, which is outwardly spaced apart from the inner wall 63 by a predetermined interval and coupled to the inner container cover 40 so as to be vertically movable, downwardly extends.

An inner wall protrusion 65 is formed on an inner periphery of the inner wall 63 and coupled to an edge of the pump top plate 53, and an outer wall protrusion 66 is formed on an inner periphery of the outer wall 64, and fastened to a fastening protrusion 47 of the inner container cover 40.

The cosmetic material exhausting holes 61 of the discharge plate 60 are formed in a radial shape over the cosmetic material diffusion space 58, and a diameter of the cosmetic material exhausting hole 61 gradually increases from a center toward an outer side thereof, such that the cosmetic material is uniformly discharged through each of the cosmetic material exhausting holes 61.

Preferably, a thickness of the discharge plate 60 is 0.5 mm to 3.0 mm.

When the thickness of the discharge plate 60 is less than 0.5 mm, the discharge plate 60 may be easily cracked or broken by an external impact or pressurization of the user, and when the thickness of the discharge plate 60 exceeds 3.0 mm, the discharge plate 60 becomes heavy, which causes the carrying inconvenience and the increase of manufacturing costs.

In addition, preferably, the diameter of the cosmetic material exhausting hole 61 of the discharge plate 60 is 0.5 mm to 1.5 mm.

When the diameter of the cosmetic material exhausting hole 61 is less than 0.5 mm, the pressure of the cosmetic material discharged by the pump 50 is increased, thus the cosmetic material suddenly bounces to the outside, and when the diameter of the cosmetic material exhausting hole 61 exceeds 1.5 mm, the diameter of the cosmetic material exhausting hole 61 is so large that external foreign matter enters and contaminates contents.

An upper portion of the discharge plate 60 is exposed to the outside to make contact with a puff P by the user, and the discharge plate 60 is formed of a ceramic material having high hardness, heat resistance and wear resistance and having antibacterial, antiseptic, heavy metal-removing and deodorizing functions.

The ceramic material forming the discharge plate 60 has high hardness, and excellent heat resistance and wear resistance, thereby enabling long-time use.

In addition, the ceramic material exerts antibacterial and antiseptic actions and heavy metal-removing and deodorizing functions to the discharge plate 60 when the cosmetic material is discharged through the discharge plate 60.

The ceramic discharge plate 60 is formed of oxide ceramics or non-oxide ceramics, in which the oxide ceramics preferably include at least one of alumina, beryllia, ceria, and zirconia, and the non-oxide ceramics preferably include at least one of carbide, boride, nitride, and silicide.

In order to have the antibacterial and antiseptic functions, metal, plant extract, or inorganic material is added to the ceramic material of the discharge plate 60, in which the metal preferably includes at least one of silver, copper, and bismuth, the plant extract preferably includes pine needle extract or mustard extract, and the inorganic material preferably includes potassium sorbate.

In addition, in order to have the heavy metal-removing function, at least one of red clay and elvan is preferably added to the ceramic material of the discharge plate 60.

In addition, in order to have the deodorizing function, at least one of charcoal, sandy soil, and clayey soil is preferably added to the ceramic material of the discharge plate 60.

A mesh 70 is additionally formed between the pump 50 and the discharge plate 60, so that the gel cosmetic material is uniformly discharged and widely diffused, and the mesh 70 is attached to the adhesion space 59 of the pump body 53 by the double-sided tape 71.

In other words, as shown in FIG. 5, the cosmetic material discharged through the pump 50 is blocked by the mesh 70 and fully filled in the cosmetic material diffusion space 58 under the mesh 70, and the cosmetic material is uniformly discharged through the mesh 70 and uniformly and widely diffused and discharged upon pumping.

In addition, because the gel cosmetic material is pumped, passes through the mesh 70, and is discharged to the outside through the cosmetic material exhausting holes 61 of the discharge plate 60, the cosmetic material discharged from the pump 50 may be prevented from suddenly splashing when the cosmetic material has low viscosity.

A method of assembling the above compact container having a discharge plate made of a ceramic material will be described as follows.

In order to assemble the compact container having a discharge plate made of a ceramic material of the present invention, as shown in FIGS. 3 and 4, at first, the inner container 30 provided therein with the cosmetic material storage membrane 33 is fitted into the outer container 10, and the cosmetic material is injected into the cosmetic material storage membrane 33.

Then, the inner container 30 is sealed by fixing the inner container cover 40 to the upper portion of the inner container 30. The first and second sealing rings 31 and 32 may be additionally coupled between the inner container 30 and the inner container cover 40 to improve the sealing performance.

Then, the pump 50 is mounted on the upper portion of the inner container cover 40. More specifically, the first pump mount protrusion 512 and the second pump mount protrusion 513 of the pump bottom plate 51, integrally formed thereon with the pump body 52 and mounted on the outer side thereof with the elastic member 56, are inserted into the first and second pump mount grooves 42 and 43 of the inner container cover 40, respectively, and the pump top plate 53 is coupled to the upper portion of the pump body 52, in which the horizontal extension piece 522 of the pump body 52 is fitted between the first extension piece 534 and the second extension piece 535 of the pump top plate 53.

Next, the discharging valve 54 is mounted on the outer side of the blocking member 531 of the pump top plate 53, the fixation member 55 is coupled to prevent the discharging valve 54 from being separated, the mesh 70 is attached to the upper end of the pump top plate 53, and the ceramic discharge plate 60 is coupled to the upper portion of the pump top plate 53 and the inner container cover 40 so as to be vertically movable.

Finally, the outer container lid 20 is hinged to one side of the outer container 10, thereby finishing the assembly of the compact container having a discharge plate made of a ceramic material of the present invention.

The above assembled compact container having a discharge plate made of a ceramic material is used as follows.

First, as shown in FIG. 5, in order to use the compact container having a discharge plate made of a ceramic material, the discharge plate 60 is pressurized by using the puff P.

When the discharge plate 60 is pressurized, the pump top plate 53 coupled to the lower portion of the discharge plate 60 downwardly moves, and the pump body 52 is pressed by the downward movement of the pump top plate 53, thus the elastic support 521 of the pump body 52 is folded and a volume of the cosmetic material temporary storage space TR is reduced.

Accordingly, the pressure generates in the cosmetic material temporary storage space TR, and the discharge pressure generates as the above pressure leads the cosmetic material in the cosmetic material temporary storage space TR to be discharged to the outside, thus the suction valve 511 of the pump bottom plate 51 closes the cosmetic material suction hole 41 of the inner container cover 40.

Simultaneously, the elastic discharge valve 54 making close contact to the outer surface of the blocking member 531 of the pump body 53 is spaced from the blocking member 531 by the cosmetic material in the cosmetic material temporary storage space TR, thus the cosmetic material discharging hole 532 is opened, and the cosmetic material is discharged through the cosmetic material discharging hole 532, and introduced into the cosmetic material diffusion space 58.

After the cosmetic material is fully filled in the cosmetic material diffusion space 58, the cosmetic material is pushed out through the mesh 70, thus the cosmetic material is simultaneously discharged through the plurality of cosmetic material exhausting holes 61 formed in the discharge plate 60.

Herein, since the cosmetic material exhausting hole 61 adjacent to the center of the discharge plate has a small diameter so that the cosmetic material is slightly discharged, however, the cosmetic material exhausting hole 61 far from the center is large, so that the cosmetic material is discharged more, as a result, the cosmetic material is evenly discharged in the same amounts throughout the cosmetic material exhausting holes 61 of the discharge plate 60.

Then, as shown in FIG. 6, when the pressurization of the discharge plate 60 is released, the elastic support 521 of the pump body 52 is restored by the elasticity of the elastic member 56 and the elastic support 521, thus a volume of the cosmetic material temporary storage space TR increases, thereby generating vacuum pressure.

Accordingly, the discharge valve 54 makes close contact with the blocking member 531 of the pump top plate 53, thus the cosmetic material discharging hole 532 is closed, and the suction valve 511 of the pump bottom plate 51 is simultaneously lifted by the vacuum pressure generated in the cosmetic material temporary storage space TR, thereby opening the cosmetic material suction hole 41 of the inner container cover 40.

Accordingly, the contents accommodated in the cosmetic material storage membrane 33 of the inner container 30 are introduced into the content material temporary storage space TR through the cosmetic material suction hole 41.

The present invention described in the above is just one embodiment for carrying out a compact container having a discharge plate made of a ceramic material, and the present invention is not limited to the embodiment. Although preferred embodiments have been proposed and set forth in the aforementioned description, it will be apparent to those having ordinary skill in the art in that many different substitutions, deformations and modifications are available within the scope without departing from the invention.

### DESCRIPTION OF REFERENCE NUMERALS

10: outer container 11: button
12: inner container accommodation space
13: mount protrusion 14: latching protrusion
20: outer container lid 21: hook 22: mirror
30: inner container 31: first sealing ring
32: second sealing ring
33: cosmetic material storage membrane 34: step
35: coupling groove 36: mount groove
37: bottom surface 38: air flow hole
40: inner container cover
41: cosmetic material suction hole
42: first pump mount groove
43: second pump mount groove
44: first upper extending protrusion wheel
45: second upper extending protrusion wheel
46: lower extending protrusion wheel
47: fastening protrusion 48: coupling protrusion
49: sealing ring insertion groove
50: pump 51: pump bottom plate 52: pump body
53: pump top plate 54: discharging valve
55: fixation member 56: elastic member
57: pump discharging space
58: cosmetic material diffusion space
60: discharge plate
61: cosmetic material exhausting hole
63: inner wall 64: outer wall
65: inner wall protrusion 66: outer wall protrusion
70: mesh 71: double-sided tape
TR: cosmetic material temporary storage space
P: puff

1. A compact container having a discharge plate made of a ceramic material, the compact container comprising:
a pump (50) for discharging a cosmetic material to an outside; and
the discharge plate (60) coupled to a top of the pump (50), formed therein with a plurality of cosmetic material outlets (61), having an upper portion exposed to the outside to make contact with a puff (P) by a user, and made of the ceramic material having high hardness, heat resistance and wear resistance.

2. The compact container of claim 1, wherein the ceramic material of the discharge plate (60) has antibacterial, antiseptic, heavy metal-removing and deodorizing functions.

3. The compact container of claim 1, wherein the discharge plate (60) has a thickness of 0.5 mm to 3.0 mm.

4. The compact container of claim 1, wherein the cosmetic material outlet (61) of the discharge plate (60) has a diameter of 0.5 mm to 1.5 mm.

5. The compact container of claim 1, wherein the cosmetic material outlet (61) of the discharge plate (60) has a diameter which gradually increases from a center to an outer side of the discharge plate (60).

6. The compact container of claim 1, wherein the ceramic discharge plate (60) made of the ceramic material of oxide ceramics or non-oxide ceramics, and
wherein oxide ceramics include at least one of alumina, beryllia, ceria, and zirconia, and the non-oxide ceramics include at least one of carbide, boride, nitride, and silicide.

7. The compact container of claim 6, wherein at least one of metal, plant extract, and inorganic material is added to the ceramic material of the discharge plate (60) to impart the antibacterial and antiseptic functions to the discharge plate (60).

8. The compact container of claim 7, wherein the metal includes at least one of silver, copper, and bismuth, the plant extract includes pine needle extract or mustard extract, and the inorganic material includes potassium sorbate.

9. The compact container of claim 6, wherein red clay or elvan is added to the ceramic material of the discharge plate (60) to impart the heavy metal-removing function to the discharge plate (60).

10. The compact container of claim 6, wherein at least one of charcoal, sandy soil, and clayey soil is added to the ceramic material of the discharge plate (60) to impart the deodorizing function to the discharge plate (60).

11. The compact container of claim 1, wherein the pump (50) is formed at a top end thereof with a cosmetic material diffusion space (58) having a radial shape.

12. The compact container of claim 1, further comprising a mesh (70) interposed between the pump (50) and the discharge plate (60).

13. The compact container of claim 12, wherein the mesh (70) is attached to a top end of the pump (50) by using a double-sided tape (71).

14. The compact container of claim 2, wherein the discharge plate (60) has a thickness of 0.5 mm to 3.0 mm.

15. The compact container of claim 2, wherein the cosmetic material outlet (61) of the discharge plate (60) has a diameter of 0.5 mm to 1.5 mm.

16. The compact container of claim 2, wherein the cosmetic material outlet (61) of the discharge plate (60) has a diameter which gradually increases from a center to an outer side of the discharge plate (60).

17. The compact container of claim 2, wherein the ceramic discharge plate (60) made of the ceramic material of oxide ceramics or non-oxide ceramics, and
wherein oxide ceramics include at least one of alumina, beryllia, ceria, and zirconia, and the non-oxide ceramics include at least one of carbide, boride, nitride, and silicide.

18. The compact container of claim 17, wherein at least one of metal, plant extract, and inorganic material is added to the ceramic material of the discharge plate (60) to impart the antibacterial and antiseptic functions to the discharge plate (60).

19. The compact container of claim 18, wherein the metal includes at least one of silver, copper, and bismuth, the plant extract includes pine needle extract or mustard extract, and the inorganic material includes potassium sorbate.

20. The compact container of claim 17, wherein red clay or elvan is added to the ceramic material of the discharge plate (60) to impart the heavy metal-removing function to the discharge plate (60).

21. The compact container of claim 17, wherein at least one of charcoal, sandy soil, and clayey soil is added to the ceramic material of the discharge plate (60) to impart the deodorizing function to the discharge plate (60).
